(19) United States
(12) Patent Application Publication
(10) Pub. No.: US 2025/0186364 A1
(43) Pub. Date: Jun. 12, 2025

(54) COMBINATION OF ANTISPASMODIC AND ANESTHETIC AGENTS FOR TOPICAL PRODUCTS AND THEIR METHOD OF USE

(71) Applicants: Priti Shanghvi Shah, Rockaway, NJ (US); Mandar V Shah, Rockaway, NJ (US)

(72) Inventors: Priti Shanghvi Shah, Rockaway, NJ (US); Mandar V Shah, Rockaway, NJ (US)

(21) Appl. No.: 18/974,349

(22) Filed: Dec. 9, 2024

Related U.S. Application Data
(60) Provisional application No. 63/608,295, filed on Dec. 10, 2023.

(51) Int. Cl. A61K 31/047; A61K 9/00; A61K 31/56; A61K 45/06; A61P 21/02

(52) U.S. Cl. CPC A61K 31/047; A61K 9/0014; A61K 31/56; A61K 45/06; A61P 21/02

(57) ABSTRACT

The present invention is directed to pharmaceutical compositions comprising one or more antispasmodic agent selected from C₄-C₈ aliphatic-1,2-diols and/or C₄-C₈ aliphatic-1,2,3-triols in combination with one or more anesthetic agent(s) to relieve the muscle spasm and/or pain associated with muscle spasm. These agents may be administered topically in one of the pharmaceutically accepted dosage forms, such as gel, cream, lotion, ointment, patch, spray, foam, film forming mixture, emulsion, microemulsion, suspension, poultice, liniment, tincture, etc. The invention also directed to a process of preparing such compositions and methods and uses thereof.

### CROSS REFERENCE TO RELATED APPLICATION

This application claims the benefit of United States Provisional application of Priti Shanghvi Shah and Mandar V Shah, application No. 63/608,295 filed on Dec. 10, 2023, having the title for COMBINATION OF ANTISPASMODIC AND ANESTHETIC AGENTS FOR TOPICAL PRODUCTS which is incorporated herein by reference in its entirety.

### FIELD OF THE INVENTION

The present disclosure provides methods for the preparation and use of pharmaceutical compositions comprising one or more antispasmodic agent(s) alone or in combination with one or more anesthetic agent(s) and their use to relieve muscle spasm and/or pain associated with muscle spasm. These compositions may be administered topically or orally and further may be combined with anti-inflammatory agents such as a steroidal or non-steroidal anti-inflammatory agent(s) or muscle relaxing agent(s).

### BACKGROUND

Muscular spasm is defined by sudden involuntary contraction of a muscle or a group of muscles. Muscle spasms are very common and can happen in any part of the body, but they tend to affect mostly to feet, hands, arms, thighs, abdomen and intercostal muscles. The common causes for muscle spasm are muscle pain, fatigue, and overuse however, other causes can be stress, anxiety etc. These muscular spasms are associated with some kind of inflammation. A muscle spasm can lead to stimulation of mechanosensitive pain receptors thereby causing a sensation of pain. Thus, pain can arise from or be due to a muscle spasm. Additionally, the spasm can indirectly stimulate the pain receptors by compressing onto blood vessels, causing ischemia in the tissue, which in turn releases pain inducing substances that stimulate pain receptors to cause pain sensations. Furthermore, a muscle spasm can cause a localized pH reduction, which can be perceived as, or which can engender pain signals. Hence, pain can be a secondary effect of a muscle spasm or muscle hypertonicity.

Ineffectively treated pain can be devastating to the person experiencing it by limiting function, reducing mobility, complicating sleep, and dramatically interfering with the quality of life.

Many times, oral non-steroidal agents (NSAIDs) treat the muscular inflammation, however, utilizing oral dosage forms to treat localized joint or muscle pain has a significant disadvantage of exposing the whole body to the drug. Topical application of anti-inflammatory agents offers the possibility of achieving local therapeutic benefit while reducing or eliminating the risk of systemic exposure and effects. As a result, anti-inflammatory agents such as a non-steroidal anti-inflammatory drug and steroids gel, cream, lotion or ointment have gained in popularity. While all anti-inflammatory agents reduce the inflammation, they do little to reduce the muscle spasm. Also, in extreme cases, oral anti-inflammatory agents are associated with side effects such as ulcers, skin rashes, itching, burning, bleeding, or holes in the stomach or intestine.

In the field of pharmaceutical preparations, topical antispasmodic agent(s) alone in combinations have already described in scientific and patent literature.

U.S. Pat. No. 6,277,892 discloses pharmaceutical compositions for topical application comprising a safe and effective amount of a pharmaceutical active, and from about 0.1% to about 10.0% of a high molecular weight cationic polymer. These compositions provide enhanced penetration of the pharmaceutical actives. These compositions can also contain one or more additional humectants/moisturizers, many of which may also be useful as actives wherein the preferred humectants/moisturizers for use in the compositions of this invention are the C₃-C₆ diols and triols. Especially preferred is the triol, glycerin.

European Patent No. EP 2,340,043 A1 discloses a composition for improved transdermal drug delivery comprising a drug, a combination of at least two penetration enhancing agents, wherein at least one of the penetration enhancing agents is selected from the group consisting of esters of saturated or unsaturated fatty acids and lower alcohols, and isoform alcohols; wherein at least one of the penetration enhancing agents is selected from the group consisting of aliphatic diols and triols; and wherein the components are present in a non-aqueous solvent system.

U.S. Pat. No. 8,236,348 discloses a dosage form wherein the dosage form is made adhesive by using a lower molecular weight hydrophilic polymer rather than by incorporation of additional polymers not contained within the wet matrix. When the dosage forms of the invention serve as transmucosal delivery systems, various carriers and additives may be incorporated as is well known in the art of transmucosal (e.g., buccal) drug delivery. Typical additives include permeation enhancers such as polyethylene glycol esters, long-chain fatty acid esters of diols and triols (e.g., glycerol monolaurate, propylene glycol monolaurate), lower alkanols, and the like.

U.S. Pat. No. 8,853,189 B2 discloses antispasmodic C₄-C₈ aliphatic-1,2-diols and C₄-C₈ aliphatic-1,2,3-triols and their use to relieve the spasms associated with pain. C₄-C₈ aliphatic-1,2-diol, a C₄-C₈ aliphatic-1,2,3-triol and or its derivatives are widely used in cosmetic industry, however use of C₄-C₈ aliphatic-1,2-diol and/or a C₄-C₈ aliphatic-1,2,3-triol in pharmaceutical industry especially for relieve muscle spasm and/or pain associated with muscle spasm is not approved or reported.

Thus, a heretofore unaddressed need exists in the industry to address the aforementioned deficiencies and inadequacies. In particular, there exists an enduring need to develop antispasmodic agent(s) in combination with anesthetic agent(s) formulation that provides pharmaceutical compositions and their use to relieve muscle spasm and/or pain associated with muscle spasm.

### SUMMARY

The present disclosure provides methods for the preparation and use of pharmaceutical compositions comprising one or more antispasmodic agent(s) alone or in combination with one or more anesthetic agent(s) and their use to relieve muscle spasm and/or pain associated with muscle spasm. These compositions may be administered topically or orally and further may be combined with anti-inflammatory agents such as a steroidal or non-steroidal anti-inflammatory agent(s) or muscle relaxing agent(s).

In another general aspect, there is provided a pharmaceutical composition comprising one or more antispasmodic agent(s) alone or in combination with one or more anesthetic agent(s).

In another general aspect, there is provided a pharmaceutical composition comprising one or more antispasmodic agent(s) alone or in combination with one or more anesthetic agent(s) to relieve the muscles spasms and/or pain associated with muscle spasm.

In another general aspect, there is provided a pharmaceutical composition comprising one or more antispasmodic agent(s) alone or in combination with one or more anesthetic agent(s) and one or more pharmaceutically acceptable excipients to relieve the muscle spasm and/or pain associated with muscle spasm.

In another general aspect, there is provided a topical pharmaceutical composition comprising one or more antispasmodic agent(s) selected from a C₄-C₈ aliphatic-1,2-diol, a C₄-C₈ aliphatic-1,2,3-triol and or its derivatives alone or in combination with one or more anesthetic agent(s) to relieve the muscle spasm/or pain associated with muscle spasm.

In another general aspect, there is provided a pharmaceutical composition comprising one or more antispasmodic agent(s) selected from a C₄-C₈ aliphatic-1,2-diol, a C₄-C₈ aliphatic-1,2,3-triol and or its derivatives alone or in combination with one or more anesthetic agent(s) selected from lidocaine, bupivacaine, prilocaine, procaine, tetracaine, cocaine, ambucaine, amolanone, amylocaine, benoxinate, benzocaine, betoxycaine, biphenamine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dycyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, propanocaine, proparacaine, propipocaine, propoxycaine, pseudococaine, pyrrocaine, ropivacaine, salicyl alcohol, tolycaine, trimecaine, zolamine, and salts thereof to relieve the muscle spasm and/or pain associated with muscle spasm.

In another general aspect, there is provided a stable pharmaceutical compositions comprising one or more antispasmodic agent(s) selected from a C₄-C₈ aliphatic-1,2-diol, a C₄-C₈ aliphatic-1,2,3-triol and or its derivatives alone or in combination with anesthetic agent(s) selected from lidocaine, bupivacaine, prilocaine, procaine, tetracaine, cocaine, ambucaine, amolanone, amylocaine, benoxinate, benzocaine, betoxycaine, biphenamine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dycyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, propanocaine, proparacaine, propipocaine, propoxycaine, pseudococaine, pyrrocaine, ropivacaine, salicyl alcohol, tolycaine, trimecaine, zolamine, and salts thereof to relieve muscle spasm and/or pain associated with muscle spasm.

In another general aspect, there is provided a pharmaceutical composition comprising one or more antispasmodic agent(s) selected from a C₄-C₈ alkane-1,2-diol and/or a C₄-C₈ alkane-1,2,3-triol and or its derivatives alone or in combination with one or more anesthetic agent(s) selected from lidocaine, bupivacaine, prilocaine, procaine, tetracaine, cocaine, ambucaine, amolanone, amylocaine, benoxinate, benzocaine, betoxycaine, biphenamine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dycyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, propanocaine, proparacaine, propipocaine, propoxycaine, pseudococaine, pyrrocaine, ropivacaine, salicyl alcohol, tolycaine, trimecaine, zolamine, and salts thereof to relieve the muscle spasm and/or pain associated with muscle spasm.

In another general aspect, there is provided a pharmaceutical composition comprising one or more antispasmodic agent(s) is a C₄-C₈ alkane-1,2-diol, selected from a 1,2-butanediol, 1,2-pentanediol, 1,2-hexanediol (HD), 1,2-heptanediol, and 1,2-octanediol and/or a C₄-C₈ alkane-1,2,3-triol, selected from 1,2,3-butanetriol, 1,2,3-pentanetriol, 1,2,3-hexanetriol, 1,2,3-heptanetriol, and 1,2,3-octanetriol alone or in combination with one or more anesthetic agent(s) selected from lidocaine, bupivacaine, prilocaine, procaine, tetracaine, cocaine and like to relieve the muscle spasm and/or pain associated with muscle spasm.

In another general aspect, there is provided a pharmaceutical composition comprising one or more antispasmodic agent(s) present in an amount of about 0.5% to about 20% w/w alone or in combination with one or more anesthetic agent(s) present in an amount of about 0.01% to about 10% w/w.

In another general aspect, there is provided a pharmaceutical composition comprising one or more antispasmodic agent(s) selected from a C₄-C₈ aliphatic-1,2-diol, a C₄-C₈ aliphatic-1,2,3-triol and or its derivatives, present in an amount of about 0.5% to about 20% w/w alone or in combination with one or more anesthetic agent(s) present in an amount of about 0.01% to about 10% w/w.

In another general aspect, there is provided a pharmaceutical composition comprising one or more antispasmodic agent(s) alone or in combination with one or more anesthetic agent(s) to relieve the muscle spasm and/or pain associated muscle spasm, further comprising an anti-inflammatory agent(s) such as a steroidal or non-steroidal anti-inflammatory agent(s), muscle relaxing agent(s) or pharmaceutically acceptable salt thereof.

In another general aspect, there is provided a pharmaceutical composition comprising one or more antispasmodic agent(s) alone or in combination with one or more anesthetic agent(s) to relieve the muscle spasm and/or pain associated with muscle spasm, wherein the pharmaceutical compositions administered topically in one of the pharmaceutically accepted dosage forms, such as gel, cream, lotion, ointment, patch, spray, foam, film forming mixture, emulsion, microemulsion, suspension, poultice, liniment, tincture, etc.

In another general aspect, there is provided a pharmaceutical composition comprising one or more antispasmodic agent(s) alone or in combination with one or more anesthetic agent(s) to relieve the muscle spasm and/or pain associated with muscle spasm, further comprising a therapeutically effective amount of an anti-inflammatory agent(s) such as a steroidal or non-steroidal anti-inflammatory agent(s), muscle relaxing agent(s) or pharmaceutically acceptable salt thereof.

In another general aspect, there is provided a method of treating or relieving muscle spasm and/or pain associated with muscle spasm in a subject in need thereof, the method comprising administering to the subject a pharmaceutical composition comprising one or more antispasmodic agent(s) alone or in combination with one or more anesthetic agent(s) and one or more pharmaceutically acceptable excipients.

In another general aspect, there is provided method of treating or relieving muscle spasm and/or pain associated with muscle spasm in a subject in need thereof, comprising one or more antispasmodic agent(s) alone or in combination with one or more anesthetic agent(s) to relieve the muscle spasm and/or pain associated with muscle spasm, further comprising a therapeutically effective amount of inflammatory agent(s) such as a steroidal or non-steroidal anti-inflammatory agent(s), muscle relaxing agent(s), or pharmaceutically acceptable salt thereof.

In another general aspect, there is provided method of the treating muscle spasms associated with torticollis, the method comprising administering to the subject a pharmaceutical composition comprising (HD) present in an amount of about 0.5% to about 20% w/w alone or in combination with lidocaine present in an amount of about 0.01% to about 10% w/w and one or more pharmaceutically acceptable excipients.

In another general aspect, there is provided method of the treating muscle spasms associated with torticollis, the method comprising administering to the subject a pharmaceutical composition comprising HD present in an amount of about 5% w/w alone or in combination with lidocaine present in an amount of about 4% w/w and one or more pharmaceutically acceptable excipients.

In another general aspect, there is provided method of the treating muscle spasms wherein the muscle spasms are associated with pain after chemotherapy or cervical myofascial pain syndrome also commonly known as tension type headache, the method comprising administering to the subject a pharmaceutical composition comprising HD present in an amount of about 5% w/w alone or in combination with lidocaine present in an amount of about 4% w/w and one or more pharmaceutically acceptable excipients.

In another general aspect, there is provided a pharmaceutical composition comprising (HD) present in an amount of about 5% w/w alone or in combination with lidocaine present in an amount of about 4% w/w and one or more pharmaceutically acceptable excipients topically applied for the treatment of muscle spasms associated with torticollis.

In another general aspect, there is provided a pharmaceutical composition comprising one or more antispasmodic agent(s) alone or in combination with one or more anesthetic agent(s) and one or more pharmaceutically acceptable excipients for once-a-day, twice-a-day and/or thrice-a-day administration.

In specific aspect, a topical pharmaceutical composition may be in the form of gel dosage form, wherein the composition comprising one or more antispasmodic agent(s) selected from a C₄-C₈ aliphatic-1,2-diol, a C₄-C₈ aliphatic-1,2,3-triol and or its derivatives alone or in combination with one or more anesthetic agent(s).

In another specific aspect, a topical pharmaceutical composition may be in the form of foam dosage form, wherein the composition comprising one or more antispasmodic agent(s) selected from a C₄-C₈ aliphatic-1,2-diol, a C₄-C₈ aliphatic-1,2,3-triol and or its derivatives alone or in combination with one or more anesthetic agent(s).

In another specific aspect, a topical pharmaceutical composition may be in the form of film forming dosage form, wherein the composition comprising one or more antispasmodic agent(s) selected from a C₄-C₈ aliphatic-1,2-diol, a C₄-C₈ aliphatic-1,2,3-triol and or its derivatives alone or in combination with one or more anesthetic agent(s).

In another general aspect, there is provided a process for preparing pharmaceutical compositions comprising one or more antispasmodic agent(s) alone or in combination with one or more anesthetic agent(s) to relieve muscle spasm and/or pain associated with muscle spasm.

In another general aspect, there is provided a process for preparing pharmaceutical compositions comprising one or more antispasmodic agent(s) selected from a C₄-C₈ aliphatic-1,2-diol, a C₄-C₈ aliphatic-1,2,3-triol and or its derivatives alone or in combination with anesthetic agent(s) selected from lidocaine, bupivacaine, prilocaine, procaine, tetracaine, cocaine, ambucaine, amolanone, amylocaine, benoxinate, benzocaine, betoxycaine, biphenamine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dycyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, propanocaine, proparacaine, propipocaine, propoxycaine, pseudococaine, pyrrocaine, ropivacaine, salicyl alcohol, tolycaine, trimecaine, zolamine, and salts thereof to relieve muscle spasm and/or pain associated with muscle spasm.

In another general aspect, there is provided a process for preparing pharmaceutical compositions comprising one or more antispasmodic agent(s) alone or in combination with anesthetic agent(s) to relieve muscle spasm and/or pain associated with muscle spasm, wherein the obtained compositions are distributed into single dose or multidose containers.

In another general aspect, there is provided a process for preparing pharmaceutical compositions comprising one or more antispasmodic agent(s) alone or in combination with one or more anesthetic agent(s) to relieve muscle spasm and/or pain associated with muscle spasm, further comprising an anti-inflammatory agent(s) such as a steroidal or non-steroidal anti-inflammatory agent(s), muscle relaxing agent(s) or pharmaceutically acceptable salt thereof.

In a specific aspect, there is provided a process for preparing a topical gel formulation of antispasmodic agent(s) in combination with anesthetic agent(s) comprising:

Phase 1: In the main tank, add the required amount of the purified water. Add carbomer 940 (polyacrylic acid) in it and heat the water to about 75° C. Stir the mixture for about 1 hour to fully hydrate the carbomer 940. After 1 hour of mixing, add antispasmodic agent(s), anesthetic agent(s), propylene glycol and glycerin. Maintain the temperature at about 75° C.

Phase II: In a secondary tank, add polyoxyl 20 cetostearyl ether, lanolin, glyceryl monostearate, sorbitan trioleate and sorbitan tristearate. Heat the mixture to about 75° C. and mix it well.

Transfer the mixture of Phase II into Phase I and mix the contents well, while maintaining the temperature of about 75° C. After about 30 minutes of mixing, add triethanolamine. Discontinue the heat and begin cooling. Once the temperature reaches about 35° C., filter the product, weigh it, add appropriate amount of isopropyl alcohol and continue stirring. Once the gel reaches room temperature, adjust the total weight with water if needed and stir well. Package the product in an appropriate container. Depending on stability of anesthetic agent, its addition may be done at a later stage of manufacturing when the product is little cool.

In another aspect, there is provided a process for preparing a topical foam formulation of antispasmodic agent(s) in combination with anesthetic agent(s) comprising:

### Preparation of Aqueous Phase:

Step 1: Add the required quantity of propylene glycol in about 70% of water in a suitable container and heat it to about 70° C.

Step 2: Add methyl and propyl parabens under stirring until dissolves completely.

Step 3: Add antispasmodic agent(s) and anesthetic agent(s) to complete the aqueous phase.

### Preparation of Oil Phase:

Step 1: Add the required quantity of cetyl alcohol, emulsifying wax and Brij in another suitable container.

Step 2: Heat the container to melt the wax and to ensure its homogeneity to complete oil phase.

Step 3: Transfer the oil phase to the aqueous phase under high shear homogenizer to form an emulsion. Add trolamine salicylate and adjust the weight by water and propellant.

In another aspect, there is provided a process for preparing a topical film forming formulation of antispasmodic agent(s) in combination with anesthetic agent(s) comprising:

Step 1: In a suitable container, take 70% of the required water and heat it to about 75° C.

Step 2: Slowly add the required quantity of Hypromellose under rigorous stirring to disperse properly

Step 3: Add the required quantity of PVP and PEG under stirring and cool the mixture to room temperature.

Step 4: Add required quantity of antispasmodic agent(s), followed by in combination with anesthetic agent(s) and continue stirring for about 3 hours.

Step 5: Add the required quantity of ethanol and Q.S. with water.

Step 6: Close the container and stir for about 30 minutes.

Step 7: Filter if needed and package in an appropriate container.

In yet another aspect, there is provided a pharmaceutical composition comprising one or more antispasmodic agent(s) alone or in combination with one or more anesthetic agent(s) and one or more pharmaceutically acceptable excipients, wherein the obtained formulation exhibits good stability throughout the shelf life as the impurities observed are well below the specified limits.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

The present invention and the manner in which it may be practiced is further illustrated with reference to the accompanying drawings wherein:

FIG. 1 is a schematic diagram of a validated muscle twitch experimental set-up in the present disclosure.

FIG. 2 is a graphical representation of muscle contractions during electrical stimulation in vehicle-treated control group and HD-treated group in the present disclosure.

FIG. 3 is a graphical representation of comparing the amplitude of contraction in vehicle-treated animals and HD-treated animals in the present disclosure.

FIG. 4 is a schematic diagram of a validated isolated mouse muscle contraction study.

FIG. 5 is a graphical representation of a frequency-dependent decrease in torque generation in muscles treated with 5% HD compared to vehicle-treated controls in the present disclosure.

FIG. 6 is a graphical representation of the effect of varying concentration of HD on the muscle contractile force as a percentage of baseline in the present disclosure.

FIG. 7 is an illustration of a Rota Rod apparatus in the present disclosure.

FIG. 8 is a graphical representation of the mean time spent on a Rota Rod apparatus after being treated with vehicle or 5% HD in the present disclosure.

FIG. 9A illustrates a six months old girl, diagnosed with torticollis is sitting on her mother's lap in the present disclosure.

FIG. 9B illustrates a six months old girl following 8 weeks of treatment with 5% w/w of HD and 4% w/w lidocaine formulation in the present disclosure.

### DETAILED DESCRIPTION OF THE EMBODIMENTS

Reference is now made in detail to the description of the embodiments as illustrated in the drawings. While several embodiments are described in the connection with these drawings, there is no intent to limit the disclosure to the embodiment or embodiments disclosed herein. The intent is to cover all alternatives, modifications, and equivalents.

It should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 35 U.S.C. § 112.

Unless otherwise indicated, the definitions and terminology used herein are intended to be applicable to all aspects of the disclosure and are not intended to be limiting in any way.

The terms “a”, “an”, and “the” as used herein are intended to include both the “singular” and “plural” forms, unless the context clearly indicates otherwise.

The term “about” as used herein, is used where measurements are understood to vary due to measurement issues or variability in populations, such as results of clinical studies. The scope of such terms will depend on the context of the element at issue and the understanding of those skilled in the art. In the absence of such guidance in the art, through relevant teachings or examples, the term “about” should be understood as meaning +/−10% of the indicated value(s).

The term “composition” as used herein, is interchangeable with formulation and refers to pharmaceutical compositions comprising one or more antispasmodic agent(s) in combination with one or more anesthetic agent(s); in a form suitable for administration to a mammal.

The term “topical formulation” as used herein denotes a composition that is suitable for topical delivery to a mammalian body, i.e., human and/or animal. A topical formulation is designed to produce a local effect at or beneath/around the application site.

The pharmaceutical compositions according to present invention may be in the form of topical delivery, oral delivery in appropriate dosage forms, and injectable dosage forms. Suitable dosage forms include but not limited to gel, lotion, foam, spray, tablets, capsules etc. In a preferred embodiment, the pharmaceutical composition(s) are administered topically.

The term “pharmaceutically acceptable excipient” as used herein, means a non-toxic, inert solid, semi-solid or liquid filler, diluent, encapsulating material or formulation auxiliary of any type. For example, the pharmaceutical compositions may include a variety of pharmaceutically acceptable excipients such as disintegrants, surfactants, emulsifiers, gelling agents, thickening agents, emollients, binders, fillers, humectants, lubricants, glidants, buffering agents, chelating agents, tonicity agents, stabilizing agents, permeation enhancers, antioxidants, pH adjusting agents and natural and synthetic waxes etc. Particular excipients include, but are not limited to, propylene glycol, Carbomer 940, triethanolamine, glycerin, polyoxy 20 cetostearyl ether, lanolin, glyceryl monostearate, sorbitan trioleate, sorbitan tristearate, isopropyl alcohol, ethanol, water, propellant, povidone, anhydrous colloidal silica, microcrystalline cellulose, sodium starch glycolate, lactose monohydrate, starch, magnesium stearate, etc.,

The term “propellant” as used herein refers to a compound effective in dispensing the active ingredients into a fine mist or foam; Preferred propellant in the present stable formulation includes, but not limited to, 1,1,1,2-tetrafluoroethane (HFC134a), and 1,1,1,2,3,3,3-heptafluoropropane. Suitable propellant also includes butane, isobutane, propane, and dimethyl ether. More preferably, the propellant is HFC134a.

The term “patient” and “subject” as used herein are interchangeable and can be taken to mean any living organism that can be treated with the pharmaceutical compositions disclosed herein. The terms “patient” and “subject” include mammals (such as primates or humans) and other animals, such as domestic animals (e.g., household pets including cats and dogs, horses, cattle, pigs, chickens, turkeys, and sheep) and non-domestic animals (such as wildlife).

The term “treating” or “treatment” as used herein, of a state, disorder or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof, (c) lessening the disease, disorder or condition or at least one of its clinical or subclinical symptoms or (d) relieving the disease, i.e., causing regression or amelioration of the state, disorder or condition or at least one of its clinical or subclinical symptoms.

The term “stable composition” as used herein, refers to any pharmaceutical compositions comprising one or more antispasmodic agent(s) in combination with one or more anesthetic agent(s) having sufficient physical and chemical stability to allow storage at a convenient temperature, such as between about 0° C. and about 40° C., for a commercially reasonable period. The term “physical stability” refers to maintenance of colour, dissolved oxygen level, headspace oxygen level, and particulate matter and the term “chemical stability” relates to formation of drug-related impurities in terms of total impurity, single maximum individual impurity and maximum individual unknown impurity. For the purpose of the present invention, chemical stability also includes maintenance of pH of the finished formulation. For pharmaceutical products, stability is required for commercially relevant times after manufacturing, such as for about 6, 12, 18 or 24 months, during which a product is kept in its original packaging under specified storage condition.

The term “shelf life” as used herein, refers to the amount of time the formulations may be stored without loss of potency and/or dissolution profile. Preferably, the shelf life refers to the amount of time the formulations may be stored without a loss of more than about 2.0%, about 5.0%, about 8.0% or about 10.0% of the potency and/or dissolution.

The pharmaceutical compositions comprising one or more antispasmodic agent(s) selected from a C₄-C₈ aliphatic-1,2-diol, a C₄-C₈ aliphatic-1,2,3-triol and or its derivatives, present in an amount of about 0.5% to about 20% w/w alone or in combination with one or more anesthetic agent(s) present in an amount of about 0.01% to about 10.0% w/w.

The one or more antispasmodic is a C₄-C₈ aliphatic-1,2-diol, a C₄-C₈ aliphatic-1,2,3-triol further administered in combination with a therapeutically effective amount of a steroidal or non-steroidal anti-inflammatory agent(s), muscle relaxing agent(s), or pharmaceutically acceptable salt thereof to relieve pain or inflammation or muscular spams or actinic keratosis or arthritis or all of these or related conditions. Suitable non-steroidal anti-inflammatory agent(s) (NSAID) for administration with the antispasmodic agent(s) selected from a C₄-C₈ aliphatic-1,2-diol, a C₄-C₈ aliphatic-1,2,3-triol and or its derivatives, but are not limited to, diclofenac, piroxicam, nimesulide, ketoprofen, ibuprofen, and methyl salicylate. Suitable steroidal anti-inflammatory agent(s) include, but are not limited to, steroidal agent(s) is selected from prednisolone, methylprednisolone, triamcinolone acetonide, cortisone, cortisone acetate, dexamethasone, dexamethasone acetate, dexamethasone tertiary butyl acetate, hydrocortisone acetate, prednisolone acetate, betamethasone acetate, betamethasone, fluticasone propionate, budesonide, tipredane, dexamethasone, beclomethasone diproprionate, prednisolone, flucinolone, mometasone furoate, rofleponide palmitate, flumethasone, flunisolide, ciclesonide, deflazacort, etc and the muscle relaxing agent(s) include but not limited to metaxalone, or cyclobenzaprine etc. Administration of NSAIDs or steroids may be by routes of administration and in dosages and formulations as are well known to effectively reduce inflammation. The NSAIDS include about 1% diclofenac; about 5% or about 10% ibuprofen; about 0.5% piroxicam; about 2.5%, about 10%, or about 20% ketoprofen; about 1%, 2%, or 3% nimesulide. For example, a pharmaceutical composition according to the invention may comprise about 1% diclofenac; or about 5% to about 10% ibuprofen; or about 0.5% piroxicam; or about 2.5%, to 20% ketoprofen; or about 1%, 2%, or 3% nimesulide. For example, diclofenac may be administered using Voltaren® gel, which contains 1% diclofenac.

The term “agent” as used herein includes a compound or mixture of compounds that, when added to a composition, produces a particular effect on the properties of the composition. It should be understood that the word “agents” referred to can also include their pharmaceutically acceptable esters, amides, prodrugs, salt forms, and solvates, and in the case of chiral molecules, their racemates or enantiopure compounds.

The term “therapeutically effective amount” as used herein, means sufficient amounts of the compositions to treat disorders, at a reasonable benefit/risk ratio applicable to any medical treatment. It is understood, however, that the attending physician within the scope of sound medical judgment can decide the total daily dosage of the compositions. The specific therapeutically effective dose level for any particular patient can depend upon a variety of factors including the disorder being treated and the severity of the disorder; activity of the specific compound employed; the specific composition employed; the age, body weight, general health and prior medical history, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved. Actual dosage levels of active ingredients in the pharmaceutical compositions can be varied so as to obtain an amount of the active compound(s) that is effective to achieve the desired therapeutic response for a particular patient and a particular mode of administration. In the treatment of certain medical conditions, repeated or chronic administration of compounds can be required to achieve the desired therapeutic response. “repeated or chronic administration” refers to the administration of compounds daily (i.e., every day) or intermittently (i.e., not every day) over a period of days, weeks, months, or longer. In particular, the treatment of chronic painful conditions may require such repeated or chronic administration of the compounds.

The formulations can be prepared by using any suitable technique, many of which are known to those skilled in the art and can be combined in any order.

The formulations disclosed herein can consist or consist essentially of the listed ingredients. If the formulations consist essentially of the listed ingredients, other ingredients may be present so long as they do not affect the stability of one or more antispasmodic agent(s) in combination with one or more anesthetic agent(s) in the formulation, for example, as measured by assay.

The previous definitions have been provided for clarity and should be interpreted as having a meaning that is consistent with their meaning in the context of the relevant art and the present disclosure and should not be interpreted in an idealized or overly formal sense unless expressly so defined herein. In the following description, numerous specific details will be taught in order to provide a thorough understanding of the disclosed subject matter. However, the presently disclosed subject matter may be practiced without these specific details, in some cases. Indeed, various modifications may be made without departing from the spirit and scope of the disclosure. The present disclosure is therefore not intended to be limited to the specific example embodiments discussed in the following description.

It is to be understood that the above description is intended to be illustrative and not restrictive. Many embodiments will be apparent to those of skill in the art upon reading the above description. The scope of the invention should, therefore, be determined with reference to the appended claims, along with the full scope of equivalents to which such claims are entitled. The disclosures of all articles and references, including patents, patent applications and publications, are incorporated herein by reference in their entirety and for all purposes.

The present disclosure provides a pharmaceutical composition and their use to relieve muscle spasm and/or pain associated with muscle spasm. In particular, pharmaceutical compositions comprising one or more antispasmodic agent(s) selected from a C₄-C₈ aliphatic-1,2-diol, a C₄-C₈ aliphatic-1,2,3-triol and or its derivatives in combination with one or more anesthetic agent(s) selected from lidocaine, bupivacaine, prilocaine and like thereof in a concentration of 0.5 to 10% and/or pharmaceutically acceptable excipients to relieve the muscle spasm and/or pain associated with muscle spasm.

The one or more antispasmodic agent(s) of the present invention is C₄-C₈ aliphatic-1,2-diols and C₄-C₈ aliphatic-1,2,3-triols. The term “C₄-C₈ aliphatic-1,2-diol,” as used herein refers to a C₄-C₈ alkane-1,2-diol, a C₄-C₈ alkene-1,2-diol, or a C₄-C₈ alkyne-1,2-diol. Representative examples of a C₄-C₈ alkane-1,2-diol include, but are not limited to, 1,2-hexanediol (HD), 1,2-heptanediol, 1,2-octanediol, 1,2-pentanediol, 1,2-butanediol, 4-methylpentane-1,2-diol, 4-methylhexane-1,2-diol, and 5-methylhexane-1,2-diol. Representative examples of a C₄-C₈ alkene-1,2-diol include, but are not limited to, 3-butene-1,2-diol, hex-4-ene-1,2-diol and hept-5-ene-1,2-diol. Representative examples of a C₄-C₈ alkyne-1,2-diol include, but are not limited to, hept-4-yne-1,2-diol and 6-methylhept-4-yne-1,2-diol. A “C₄-C₈ aliphatic-1,2,3-triol,” as used herein refers to a C₄-C₈ alkane-1,2,3-triol, a C₄-C₈ alkene-1,2,3-triol, or a C₄-C₈ alkyne-1,2,3-triol. Representative examples of a C₄-C₈ alkane-1,2,3-triol include, but are not limited to, 4-methylpentane-1,2,3-triol, 1,2,3-hexanetriol, 1,2,3-heptanetriol, 1,2,3-octanetriol, 1,2,3-pentanetriol, and 1,2,3-butanetriol. Representative examples of a C₄-C₈ alkene-1,2,3-triol include, but are not limited to, 4,5-dideoxy-D-erythro-pent-4-enitol (pent-4-ene-1,2,3-triol), hex-4-ene-1,2,3-triol and hept-4-ene-1,2,3-triol. Representative examples of a C₄-C₈ alkyne-1,2,3-triol include, but are not limited to, hept-4-yne-1,2,3-triol and oct-6-yne-1,2,3-triol. The compositions of the present invention preferably comprise of HD.

C₄-C₈ aliphatic-1,2-diols and C₄-C₈ aliphatic-1,2,3-triols are available from commercial sources or may be synthesized using methods and techniques well known in the art.

The one or more anesthetic agent(s) of the present invention is selected from but not limited to lidocaine, bupivacaine, prilocaine, procaine, tetracaine, cocaine, ambucaine, amolanone, amylocaine, benoxinate, benzocaine, betoxycaine, biphenamine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dycyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, propanocaine, proparacaine, propipocaine, propoxycaine, pseudococaine, pyrrocaine, ropivacaine, salicyl alcohol, tolycaine, trimecaine, zolamine and salts thereof. In certain embodiment, the one or more anesthetic agent is lidocaine, such as in the form of lidocaine hydrochloride. Generally, the pharmaceutical compositions used to relieve or treat muscle spasm and/or pain associated with muscle spasm have about 0.5 to about 20.0% of one or more antispasmodic agent(s) selected from a C₄-C₈ aliphatic-1,2-diol, a C₄-C₈ aliphatic-1,2,3-triol and or its derivatives alone or in combination with one or more anesthetic agent(s) as described herein. In certain embodiments, the concentrations of antispasmodic present in the composition is about 0.5% to about 20.0%, about 1.0% to about 15.0%, about 1.0% to about 10.0%, about 1.0%, about 5.0%, about 10.0%, about 15.0%, or about 5.0% and the concentration of anesthetic agent(s) is about 0.01% to about 10.0% w/w, about 0.5% to about 10.0%, about 1.0% to about 8.0%, about 1.0%, about 2.0%, about 3.0%, about 4.0%, or about 5.0%.

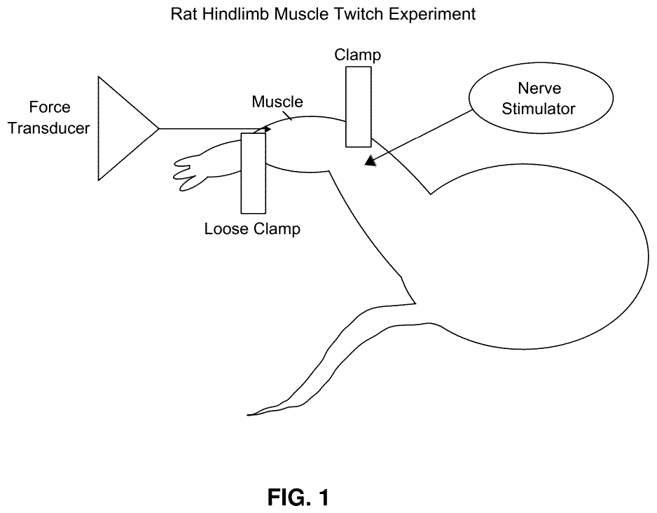

FIG. 1 is a schematic diagram of a validated muscle twitch experimental set-up in the present disclosure. Here anesthetized Sprague-Dawley rats were prepared for muscle contraction assessment of their hindlimbs. The hind limbs were shaved and secured at the knee and ankle joints to prevent movement. The distal tendon of the targeted muscle was connected to a high-sensitivity force transducer to measure contractile force with precision. Electrical stimulation was delivered via electrodes positioned on the sciatic nerve to evoke muscle contractions. Vehicle (control study) or 5% w/w of HD was topically applied to the dermal area corresponding to the muscle group under study, ensuring proximity for local diffusion and effect.

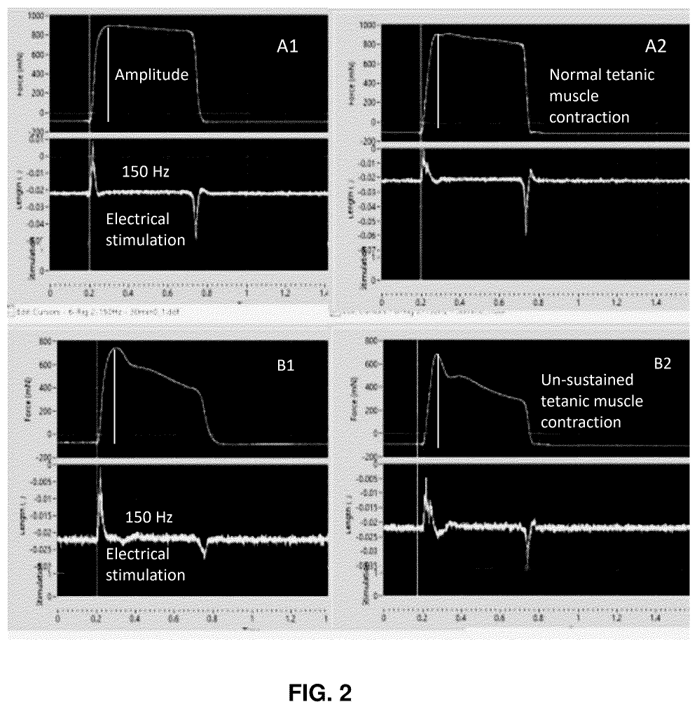

FIG. 2 is a graphical representation of muscle contractions during electrical stimulation in vehicle-treated control group, A1 and A2; and HD-treated group, B1 and B2 in the present disclosure. Upper part of the panel A1 and A2 are displaying muscle contraction in vehicle-treated group and in the same panel, lower portion is showing duration of electrical stimulation at 150 Hz. In the vehicle-treated group, the mean force output remained consistent throughout the electrical stimulus period, with no significant decrease in tetanic tension, suggesting unimpeded excitation-contraction coupling, indicative of normal muscle function.

The upper part of the panel B1 and B2 are showing muscle contraction of the HD-treated group and the lower part of the panel B1 and B2 are showing the duration of electrical stimulation at 150 Hz. HD-treated group showing initial muscle contraction followed by a rapid decline in contractile force, unable to maintain tetanic contraction throughout the stimulus period. The mean percentage decline in force output from initial peak contraction was quantitatively significant compared to the vehicle group, with a time-dependent reduction in contractile ability observed post-application of HD.

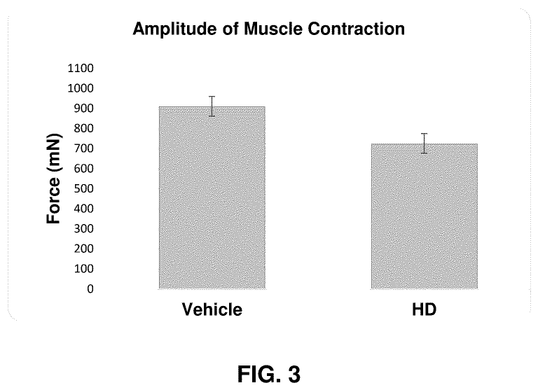

FIG. 3 is a graphical representation of comparing the amplitude of vehicle-treated animals and HD-treated animals in the present disclosure. There was a significant reduction in amplitude of contraction, from 900 mN in the vehicle-treated group (control) to 750 mN in the HD-treated group, which is statistically significant at p<0.05.

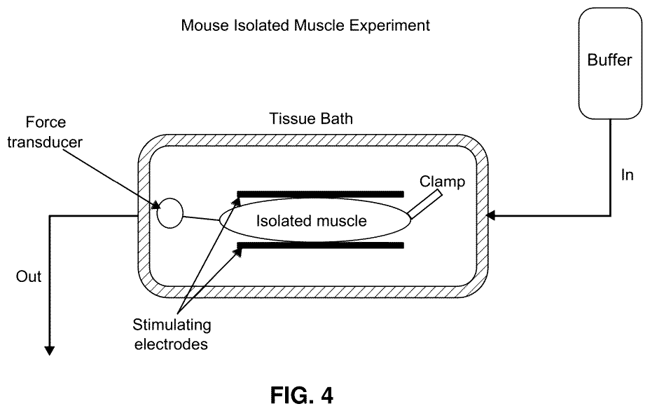

FIG. 4 is a schematic diagram of a validated isolated mouse muscle contraction study. Muscles were anchored at one end to the chamber floor and connected at the other end to a force transducer. After establishing a baseline response to the electrical field stimulation, HD was added to the buffer at concentrations ranging from 0.01% w/w to 1% w/w, and the impact on muscle contractile force was recorded over time at the frequency of 150 Hz. Additionally, muscles were subjected to varying frequencies of electrical stimulation to evaluate the frequency-dependent effects of HD.

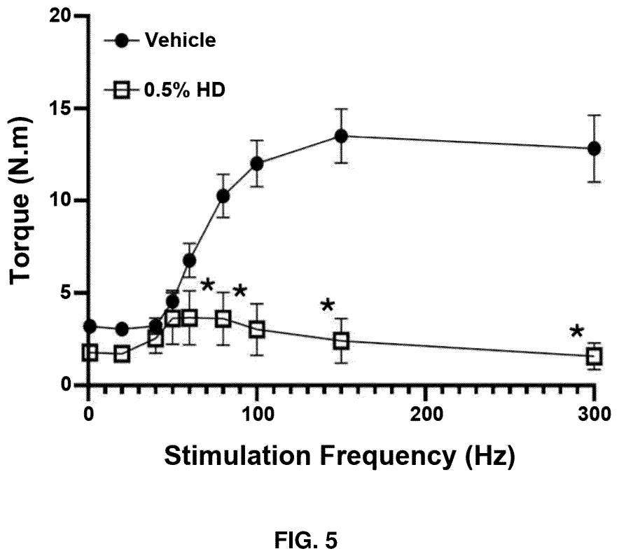

FIG. 5 is a graphical representation of a frequency-dependent decrease in torque generation in muscles treated with 0.5% HD compared to vehicle-treated controls in the present disclosure. Torque (N.m) is plotted against stimulus frequency (Hz) for both vehicle-treated and HD-treated muscle samples. Force-frequency relationship reveals that inhibition is more complete at higher frequencies of stimulation, consistent with a use-dependent mechanism of action, suggesting that HD is more effective during conditions of muscle spasm, potentially reducing the likelihood of side effects during normal muscle function. A time-control showed no loss of force (data not shown) over the same duration. In each group, n=5; *=p<0.01 vs. vehicle by two-way ANOVA with Holm-Sidak, post-hoc.

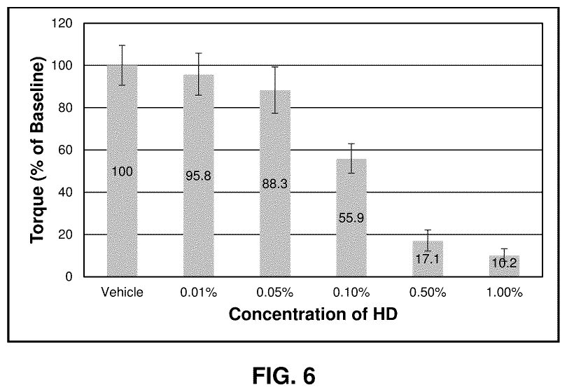

FIG. 6 is a graphical representation of the effect of varying concentration of HD on the muscle contractile force as a percentage of baseline in the present disclosure. Muscles were super fused with increasing concentrations of HD and contractile response was quantitatively measured. Data reveal that 1% HD caused an approximately 89.8% reduction in contractile force, while 0.1% HD resulted in about a 44% reduction compared to vehicle controls. No significant inhibitory effect on muscle contraction was observed at 0.01% HD.

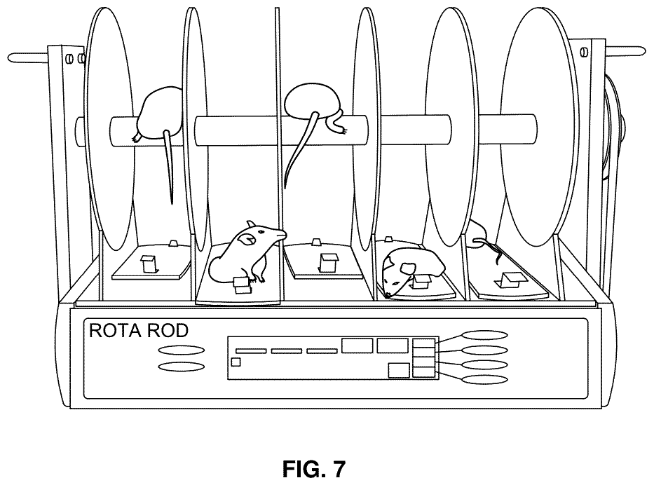

FIG. 7 is an illustration of a Rota Rod apparatus in the present disclosure. Once the mice were trained, the rod was rotated at a constant speed of 20 rpm. Then the mice were placed on the rotating rod and length of time they could stay on the rotating rod was measured in seconds.

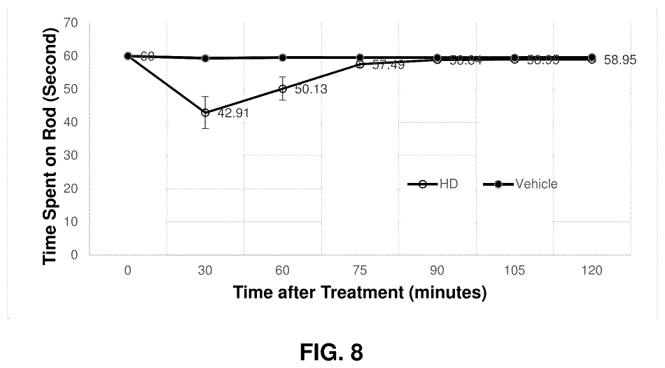

FIG. 8 is a graphical representation of the mean time spent on a Rota Rod apparatus after being treated with vehicle or 5% HD in the present disclosure. Mice treated with vehicle managed to stay on the rota rod for the full 60 seconds at all-time points after the treatment. While the mice treated with 5% HD, showed significant decrease in the time spent on rota rod after 30 and 60 minutes post treatment, showing the muscle relaxant effect of HD.

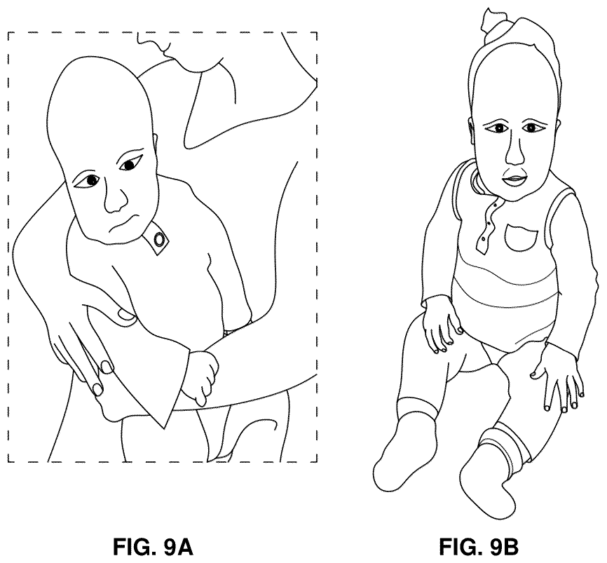

FIG. 9A illustrates a six months old girl, diagnosed with torticollis is sitting on her mother's lap in the present disclosure.

FIG. 9B illustrates a six months old girl following 8 weeks of treatment with 5% w/w of HD and 4% w/w lidocaine formulation in the present disclosure. After 8 weeks of treatment, three times a week with 5% w/w of HD and 4% w/w lidocaine formulation, girl's neck muscles started to relax normally and torticollis got resolved.

### EXAMPLES

The present invention provides various embodiments of a topical pharmaceutical composition formulated for application as a gel, foam, or film. Each embodiment disclosed herein describes specific configurations, concentrations, and optional components that may be incorporated into the composition to enhance its efficacy, stability, or application properties. These embodiments are intended to illustrate the invention and are not exhaustive; they may be combined or modified in various ways to create additional embodiments within the scope of the invention.

Below Tables provide a listing of exemplary ingredients/components suitable for an exemplary formulation of the composition of the invention and a desired weight/weight percentage for those ingredients/components.

TABLE 1
Topical Gel Composition

| Sr. No | Ingredient | % w/w |
|---|---|---|
| 1 | Lidocaine hydrochloride | 1.0 to 4.0 |
| 2 | 1,2-Hexanediol | 2.5 to 10.0 |
| 3 | Propylene glycol | 3.0 to 7.0 |
| 4 | Carbomer 940 | 1.3 to 2.0 |
| 5 | Triethanolamine | 0.5 to 1.0 |
| 6 | Glycerin | 2.0 to 8.0 |
| 7 | Polyoxyl 20 cetostearyl ether | 0.5 to 1.5 |
| 8 | Lanolin | 0.5 to 2.0 |
| 9 | Glyceryl monostearate | 1.5 |
| 10 | Sorbitan trioleate | 1.0 |
| 11 | Sorbitan tristearate | 0.5 |
| 12 | Isopropyl alcohol | 25.0 |
| 13 | Purified water | QS |

TABLE 2
Topical Foam Composition

| Sr. No | Ingredient | % w/w |
|---|---|---|
| 1 | Lidocaine hydrochloride | 0.5 to 5.0 |
| 2 | 1,2-Hexanediol | 2.5 to 10 |
| 3 | Propylene Glycol | 5 to 15 |
| 4 | Cetyl Alcohol | 0.5 to 1.5 |
| 5 | Trolamine Salicylate | 0.1 to 0.5 |
| 6 | Emulsifying Wax | 1.0 to 2.0 |
| 7 | BRIJ 76 | 0.2 to 0.8 |
| 8 | Methylparaben | 0.05 to 0.15 |
| 9 | Propylparaben | 0.05 |
| 10 | Purified Water | QS |

TABLE 3
Topical Film Forming Composition

| Sr. No | Ingredient | % w/w |
|---|---|---|
| 1 | Lidocaine hydrochloride | 0.5 to 4.0 |
| 2 | 1,2-Hexanediol | 2.5 to 10.0 |
| 3 | Polyvinyl pyrrolidone (PVP) | 10.0 to 15.0 |
| 4 | Hypromellose | 0.75 to 1.25 |
| 5 | Polyethylene glycol (PEG) | 1.0 to 2.0 |
| 6 | Ethanol | 30 to 40 |
| 7 | Purified Water | QS to 100% |

### Example 1: 1.2 Hexanediol and Lidocaine Hydrochloride Topical Gel Formulation

| Sr. No | Ingredient | % w/w |
|---|---|---|
| 1 | Lidocaine hydrochloride | 1.0 to 4.0 |
| 2 | 1,2-Hexanediol | 5.0 |
| 3 | Propylene glycol | 5.0 |
| 4 | Carbomer 940 | 1.3 to 2.0 |
| 5 | Triethanolamine | 0.7 to 1.0 |
| 6 | Glycerin | 5.0 |
| 7 | Polyoxyl 20 cetostearyl ether | 1.0 |
| 8 | Lanolin | 1.0 |
| 9 | Glyceryl monostearate | 1.5 |
| 10 | Sorbitan trioleate | 1 |
| 11 | Sorbitan tristearate | 0.5 |
| 12 | Isopropyl alcohol | 25 |
| 13 | Purified water | QS |

Manufacturing Process: A manufacturing process of the above exemplary composition has the following steps:

### Preparation of Phase I:

In the main tank, add the required amount of the purified water. Add carbomer 940 in it and heat the water to about 75° C. Stir the mixture for about 1 hour to fully hydrate the carbomer 940. While carbomer is hydrating consider preparing Phase II. After about 1 hour of mixing, add 1,2-hexanediol, propylene glycol and glycerin. Maintain the temperature at about 75° C.

### Preparation of Phase II:

In a secondary tank, add polyoxyl 20 cetostearyl ether, lanolin, glyceryl monostearate, sorbitan trioleate and sorbitan tristearate. Heat the mixture to about 75° C. and mix it well.

Transfer the mixture of Phase II into the main tank containing Phase I and mix the contents rigorously with a high shear mixer, while maintaining the temperature of about 75° C. After about 30 minutes of mixing, add triethanolamine. Discontinue the heat and begin cooling. Once the temperature reaches 40° C., weigh the contents and add water to adjust weight if needed. Add lidocaine hydrochloride and continue to stir the product. After complete dissolution, filter the contents. Weigh the contents after the filtration and add appropriate quantity of isopropyl alcohol and continue stirring. Once the gel reaches room temperature of 25° C. Package the product in an appropriate container.

### Example 2: Lidocaine Hydrochloride, 1,2 Hexanediol, and Diclofenac Sodium Topical Gel Formulation

| Sr. No | Ingredient | % w/w |
|---|---|---|
| 1 | Lidocaine hydrochloride | 1.0 to 4.0 |
| 2 | 1,2-Hexanediol | 5.0 |
| 3 | Diclofenac Sodium | 1.0 |
| 4 | Propylene glycol | 5.0 |
| 5 | Carbomer 940 | 1.3 to 2.0 |
| 6 | Triethanolamine | 0.7 to 1.0 |
| 7 | Glycerin | 5.0 |
| 8 | Polyoxyl 20 cetostearyl ether | 1.0 |
| 9 | Lanolin | 1.0 |
| 10 | Glyceryl monostearate | 1.5 |
| 11 | Sorbitan trioleate | 1 |
| 12 | Sorbitan tristearate | 0.5 |
| 13 | Isopropyl alcohol | 25 |
| 14 | Purified water | QS |

Manufacturing Process: A manufacturing process of the above exemplary composition has the following steps:

### Preparation of Phase I:

In the main tank, add the required amount of the purified water. Add carbomer 940 in it and heat the water to about 75° C. Stir the mixture for about 1 hour to fully hydrate the carbomer 940. While carbomer is getting hydrated, consider preparing Phase II. After about 1 hour of mixing, add 1,2-hexanediol, propylene glycol and glycerin. Maintain the temperature at about 75° C.

### Preparation of Preparation of Phase II:

In a secondary tank, add polyoxyl 20 cetostearyl ether, lanolin, glyceryl monostearate, sorbitan trioleate and sorbitan tristearate. Heat the mixture to about 75° C. and mix it well.

Transfer the mixture of Phase II into the main tank containing Phase I and mix the contents rigorously with a high shear mixer. while maintaining the temperature of about 75° C. After about 30 minutes of mixing, add triethanolamine. Discontinue the heat and begin cooling. Once the temperature reaches 40° C. add the required quantity of lidocaine hydrochloride and diclofenac one after the other ensuring its complete dissolution; continue stirring. Weigh the contents and add water if needed to achieve the desired weight. Then filter the contents. Weigh the contents after the filtration and add appropriate quantity of isopropyl alcohol and continue stirring. Once the gel reaches room temperature of 25° C. Package the product in an appropriate container.

### Example 3: 1,2 Hexanediol and Lidocaine Hydrochloride Topical Foam Formulation

| Sr. | | |
|---|---|---|
| No | Ingredient | w/w % |
| 1 | Lidocaine hydrochloride | 0.5 to 5.0 |
| 2 | 1,2-Hexanediol | 5.0 to 6.0 |
| 3 | Propylene Glycol | 10.0 |
| 4 | Cetyl Alcohol | 0.8 |
| 5 | Trolamine Salicylate | 0.2 |
| 6 | Emulsifying Wax | 1.5 |
| 7 | BRIJ 76 | 0.5 |
| 8 | Methylparaben | 0.1 |
| 9 | Propylparaben | 0.05 |
| 10 | Purified Water | QS |
| 11 | Tetrafluoroethane (HFC 134a) propellant | QS |

Manufacturing Process: A manufacturing process of the above exemplary composition has the following steps:

In a suitable container, take about 70% of the required water. Add propylene glycol and heat it to a temperature of about 70° C. Add methyl and propyl parabens under stirring until the parabens dissolve completely. To this, add HD and ensure its dissolution. This is the complete aqueous phase.

In a separate container, take the required quantity of cetyl alcohol, emulsifying wax and Brij 76. Heat the container to about 70° C. to melt the wax and stir to ensure the homogeneity of mixture. This is the complete oil phase.

Add oil phase to the aqueous phase maintained at 70° C. under stirring at high speed to form a coarse emulsion. Cool the emulsion to room temperature, then add trolamine salicylate and lidocaine hydrochloride. Stir to ensure their dissolution. Adjust the total weight of the emulsion by purified water if needed. Pass the complete emulsion through appropriate high shear homogenizer. Above formulation should be 90% by total weight of the final formulation and remaining 10% of the weight to be made up by propellant, HFC 134a during packaging. Package the product in an appropriate container.

### Example 4: 1,2 Hexanediol and Lidocaine Hydrochloride Film Forming Formulation

| Sr. | | |
|---|---|---|
| No | Ingredient | w/w % |
| 1 | Lidocaine Hydrochloride | 0.5 to 4.0 |
| 2 | 1,2-Hexanediol | 5.0 |
| 3 | Polyvinyl Pyrrolidone (PVP) | 10.0 to 15.0 |
| 4 | Hypromellose | 1.0 |
| 5 | Polyethylene glycol (PEG) | 1.5 |
| 6 | Ethanol | 30 to 40 |
| 7 | Purified Water | QS |

Manufacturing Process: A manufacturing process of the above exemplary composition has the following steps:

In a suitable container, take about 70% of the required water and heat it to about 75° C. Add slowly the required quantity of hypromellose under rigorous stirring until dispersed uniformly. Add the required quantity of PVP and PEG; stir it for about 30 minutes. Discontinue the heat and allow the mixture to cool at room temperature. Add required quantity of 1,2-hexanediol, followed by lidocaine hydrochloride and stir for about 3 hours. Add the required quantity of ethanol and QS with water. Filter the product and package it. Tightly close the container to prevent the evaporation of alcohol. Stir the resulting composition for about 30 minutes. Filter the solution through suitable filter and package in an appropriate container.

Evaluation of 1,2-Hexanediol (HD) for Skeletal Muscle Relaxant Activity and its Combination with Lidocaine to Control Pain:

To even further exemplify and illuminate aspects of the invention, the following description of illustrative applications of particular aspects of the invention is provided. These are meant to exemplify some aspects of the invention but should not be used to limit the scope of the invention in any manner.

The preliminary investigations into HD as a skeletal muscle relaxant have yielded compelling evidence of its efficacy and some indications regarding its mechanism of action across various experimental models. These studies form the basis for our hypothesis that HD can significantly contribute to the treatment of muscle spasms without the side effects associated with current pharmacological treatments. The preliminary data unequivocally demonstrate HD's potential as a highly effective and use-dependent skeletal muscle relaxant. Its ability to significantly reduce muscle contractility without the side effects typical of current treatments positions HD as a promising therapeutic agent. Further, its combination with Lidocaine is very useful in controlling pain, allowing manual therapy, as shown in a young torticollis patient. The combination of Lidocaine with HD can be useful in many situations, such as torticollis, or muscular spasm after chemotherapy or in cervical myofascial pain syndrome, commonly known as tension type headache.

### Rat Hind Limb Muscle Twitch Study

### A. Study Design & Methodology

A study was performed to evaluate the effects of HD on muscle contractility using a validated in vivo rat model. The experimental setup was meticulously designed to measure the contractile force of the hind limb muscle in response to electrical stimulation of the sciatic nerve, providing a quantitative assessment of muscle relaxation and contraction dynamics in the presence of HD. Anesthetized Sprague-Dawley rats were used for this study, ensuring minimal variability in response to HD application. Hind limbs were shaved and then immobilized at the knee and ankle joints to isolate the effect of the targeted muscle group. The distal tendon of the targeted muscle was connected to a high-sensitivity force transducer for accurate measurement of contractile force. An electrical stimulation was applied via electrodes strategically placed on the sciatic nerve, designed to evoke muscle contractions with controlled frequency to determine amplitude, and duration parameters. A specific concentration of 5% w/w of HD in lubricating lotion, prepared on the same day, was topically applied to the skin overlaying the muscle group under study and the control group received a vehicle (lubricating lotion) treatment under identical conditions.

### B. Results and Discussion

Control Group Response: The vehicle-treated group (FIGs. A1 and A2) exhibited consistent muscle contractions throughout the electrical stimulation period. The mean force output was stable, indicating normal excitation-contraction coupling without the influence of HD.

HD Treated Group Response: In stark contrast, the HD-treated group (FIGs. B1 and B2) showed slightly lower initial contraction response that rapidly declined, failing to maintain tetanic tension. The mean percentage decline in force output from the initial peak was quantitatively significant when compared to the vehicle-treated group. This decline was observed to be time-dependent, with the contractile ability reducing significantly post-application of HD. A detailed statistical analysis was conducted to compare the amplitude and duration of muscle contraction between the control and HD-treated groups. The analysis revealed a statistically significant reduction (p<0.05) in the duration of muscle contraction in the HD-treated rats. This reduction correlated with both the duration and frequency of the applied electrical stimulus, suggesting a potent use-dependent action of HD on muscle contractility. The quantitative and technical analysis of the muscle contraction dynamics in the presence of HD provides compelling evidence of its use-dependent inhibitory effect on skeletal muscle contractility.

Results are shown in FIGS. 1-3. FIG. 1 shows a schematic experimental set-up, where anesthetized Sprague-Dawley rats were prepared for muscle contraction assessment.

FIG. 2 illustrates that the vehicle-treated control group (FIGs. A1 and A2) displaying sustained muscle contractions during continuous electrical stimulation, indicative of normal muscle function. The mean force output remained consistent throughout the electrical stimulus period, with no significant decrease in tetanic tension, suggesting unimpeded excitation-contraction coupling. HD-treated group (FIGs. B1 and B2) is showing initial muscle contraction followed by a rapid decline in contractile force, unable to maintain tetanic contraction throughout the stimulus period. The mean percentage decline in force output from initial peak contraction was quantitatively significant compared to the vehicle group, with a time-dependent reduction in contractile ability observed post-application of HD.

FIG. 3 illustrates graphical representation comparing the amplitude (maximum contraction force in the previous graph) of vehicle-treated animals and HD-treated animals. There was a significant reduction in amplitude of contraction, from 900 mN in vehicle-treated group to about 750 mN in HD-treated group. Quantitative analysis of contraction duration revealed a statistically significant reduction (p<0.05) in the HD-treated group, with the effect magnitude correlating with stimulus duration and frequency. Control muscles exhibit no reduction in the ability to sustain contractions, whereas HD-treated muscles show a marked decline, supporting the potential therapeutic benefit of HD in conditions characterized by muscle hyperactivity. Quantitative measures of contraction force and duration provide compelling evidence for HD's myogenic mechanism of action, potentially via ion channel modulation or interference with excitation-contraction.

### Isolated Mouse Muscle Study

### A. Study Design & Methodology

This subsequent study was aimed to elucidate the specific effects of HD on isolated muscle tissue from mice, to quantify its potential use as a muscle relaxant. The study's design allowed for the controlled assessment of muscle contractility in a physiological buffer, minimizing external variables present in in vivo models. Muscle Isolation: The mouse extensor digitorum longus muscles were carefully dissected and mounted in a tissue bath super-fused with oxygenated physiological buffer, ensuring tissue viability throughout the experiment (FIG. 4). Baseline Measurement: Initial contractile responses were established via electrical stimulation at various frequencies, setting a comparative baseline for subsequent HD application effects. HD Application: Differing concentrations of HD (ranging from 0.01% to 1%) were introduced to the buffer. The muscle's contractile force (amplitude, which is the maximum contractile force) was determined to assess HD's concentration-dependent effects at a fixed frequency of 150 Hz (FIG. 6). Stimulation Frequency Adjustment: To explore the frequency-dependent nature of HD's action, muscles underwent varied frequencies of electrical stimulation at a fixed concentration of 0.5% of HD. This protocol aimed to simulate conditions of sporadic muscle spasms.

### B. Results and Discussion

Concentration-Dependent Effects: The study identified a pronounced decrease in muscle contractility with increasing HD concentrations at the fixed frequency of 150 Hz. Notably, a 1% HD solution led to an approximate 89.8% reduction in contractile force, a stark contrast to the minimal effects observed at the lowest concentration (0.01%). Frequency-Dependent Inhibition: Higher electrical stimulation frequencies revealed a more significant inhibitory effect of HD on contractile force of the muscle. This observation supports the hypothesis that HD's muscle relaxant action is use-dependent, becoming more pronounced under conditions mimicking muscle hyperactivity. The results underwent statistical analysis, comparing the contractile responses across different HD concentrations and stimulation frequencies to the control (vehicle-treated) samples. T-tests confirmed the significant, dose-dependent reduction in muscle contractility with HD treatment, achieving statistical significance (*p<0.05) at higher stimulation frequencies. The detailed investigation into the concentration and frequency-dependent effects of HD on isolated mouse muscle tissue presents a compelling case for its concentration and use-dependent mechanism of action. These findings, characterized by significant reductions in muscle contractility, align with the hypothesis that HD could be an effective muscle relaxant, in conditions characterized by increased muscle activity or spasms.

### Rotarod Performance Study in Hairless Mice

### A. Study Design & Methodology

This study was conducted to assess the muscle relaxant potential of HD using a Rotarod test. The study demonstrated that topical application of HD significantly reduced the time mice remained on the Rotarod post-treatment, confirming its efficacy as a muscle relaxant.

The Rotarod study was to investigate the skeletal muscle relaxant effects of HD, specifically its impact on motor coordination and balance in hairless mice. The study was structured to assess both the kinetics of HD's effect over time and its efficacy in inducing muscle relaxation. Efficacy of HD was determined post-treatment at intervals of 15, 30, 60, 75, 90 minutes, and another to evaluate the efficacy, particularly noting the peak effect at 30 minutes post-application. Control and Treatment Groups: Each stage included a control group receiving vehicle (Aloe Vera Gel) and a test group treated with HD, 5% w/w HD (0.5 g) in the vehicle (9.5 g Aloe Vera Gel), prepared right before the study. The topical treatment was applied to the thighs of all four limbs for each mouse.

### B. Results and Discussion

Kinetics of Effect: Demonstrated a significant time-dependent reduction in time spent on the Rotarod for HD-treated groups compared to vehicle controls. The most notable muscle relaxant activity was observed at 30 minutes post-treatment in preliminary experiments (data not shown). Statistically significant effects were noted at 30- and 60-minutes post-treatment. A recovery trend was observed by 75 minutes, indicating the transient nature of HD's muscle relaxant effect. Importantly, the complete recovery of all mice from the muscle relaxant effects of HD suggests its safety and absence of long-term detrimental impact. This demonstrates HD's potential as a therapeutic agent without lasting adverse effects on muscle function. Statistical analysis was conducted using Student's t-tests to compare treated and control groups. Significance was determined at p<0.05, underscoring the reproducibility and reliability of the observed effects. The Rotarod study's detailed quantitative analysis provides robust evidence of HD's efficacy as a muscle relaxant. Rotarod apparatus used in study is shown in FIG. 7. FIG. 8 illustrates Time-Dependent Effect of HD a Motor Coordination in Hairless Mice. The graph depicts the mean time (+SD) spent on the Rota Rod at different time intervals after topical application of 5% w/w of HD. Mice treated with the vehicle sustained their balance for the full duration (60 seconds) across all time points, while those treated with HD showed a significant decrease in balance time at 30 and 60 minutes, indicating a muscle relaxant effect. This is because of inability of the rat hind limb to contract normally. Importantly, all mice recovered from the muscle relaxant effect.

### Effectiveness in Torticollis/Congenital Torticollis:

Congenital Torticollis is a neuromuscular disorder characterized by the involuntary, sustained contraction of neck muscles, presents a significant therapeutic challenge. The condition leads to a twisted or abnormal neck posture and can cause chronic pain, reduced quality of life, skeletal muscle (sternocleidomastoideole) deformities, and potential substantial long-term disability. In US, torticollis affects approximately 3% of the newborn babies annually. In many cases it resolves itself. However, when the torticollis does not resolve itself, the existing treatment modalities range from pharmacotherapy to physical therapy to fitting of the helmet to botulinum toxin injections, offer limited relief and are frequently associated with adverse side effects such as Erb's Palsy. Further, it necessitates ongoing clinical interventions, highlighting a significant unmet medical need. Surgery (cutting the muscle) is a treatment option for non-responsive patients in extreme cases. This orphan condition translates into 400,000 healthcare visits each year, making it a significant burden on the healthcare system. The US cervical dystonia therapeutics market size was USD 472.09 million in 2021, as per our research, the market is expected to reach USD 747.41 million in 2031, exhibiting a CAGR of 4.7% during the forecast period. This financial burden does not account for the indirect costs, which include lost productivity (for the parents), and prolonged disability (for children).

A 5% w/w of HD and 4% w/w of lidocaine formulation similar to the one, shown in Example 1 was prepared. It was topically applied on a 6 months old girl diagnosed with torticollis. This young girl could not handle normal manipulative physical therapy, as it was too painful for her. Presence of lidocaine was critical, as it allowed the girl to handle manipulative physical therapy and HD relaxed the muscles. The girl was treated thrice a week for 8 weeks with a formulation similar to one presented in Example 1 consisting of 5% w/w of HD and 4% w/w lidocaine. Lidocaine numbs the pain, allowing the treatment while HD relaxes the muscles. After 8 weeks of treatment, girl's neck muscles started to relax normally and torticollis got resolved. Notably, the therapeutic effect has been sustained, with the patient remaining symptom-free for several months post-treatment, effectively leading a normal life without the need for surgical intervention. Before and after picture of this young girl are shown in FIG. 9. This case underlines the potential of a non-invasive cure for torticollis.

Based on the above results it can be concluded that present invention distinguishes itself by combining anesthetic agents with antispasmodic compounds to create more effective compositions for relieving the muscle spasm and/or pain associated with muscle spasm, offering improved skin penetration and dosing flexibility, not present in previous inventions. These innovative features enhance the therapeutic efficacy and versatility of the formulation, which is crucial in addressing muscle spasms and associated conditions.

Although exemplary embodiments have been shown and described, it will be clear to those of ordinary skill in the art that a number of changes, modifications, or alterations to the disclosure as described may be made. All such changes, modifications, and alterations should therefore be seen as within the scope of the disclosure.

What is claimed is:

1. A method of treating or relieving muscle spasm and/or pain associated with muscle spasm comprising:
administering, to a patient in need thereof, a therapeutically effective amount of a pharmaceutical composition comprising one or more antispasmodic agent(s) selected from C₄-C₈ aliphatic-1,2-diol and/or a C₄-C₈ aliphatic-1,2,3-triol in combination with one or more anesthetic agent(s) and one or more pharmaceutically acceptable excipients.

2. The method of claim 1, wherein the one or more antispasmodic agent(s) present in an amount of about 0.5% to about 20% w/w.

3. The method of claim 1, wherein the one or more antispasmodic agent(s) selected from a C₄-C₈ alkane-1,2-diol and/or a C₄-C₈ alkane-1,2,3-triol.

4. The method of claim 3, wherein the one or more antispasmodic agent(s) is a C₄-C₈ alkane-1,2-diol and or C₄-C₈ alkane-1,2,3-triol.

5. The method of claim 4, wherein the one or more antispasmodic agent(s) is a C₄-C₈ alkane-1,2-diol, selected from a 1,2-butanediol, 1,2-pentanediol, 1,2-hexanediol, 1,2-heptanediol, and 1,2-octanediol.

6. The method of claim 5, wherein the one or more antispasmodic agent(s) is a C₄-C₈ alkane-1,2,3-triol, selected from 1,2,3-butanetriol, 1,2,3-pentanetriol, 1,2,3-hexanetriol, 1,2,3-heptanetriol, and 1,2,3-octanetriol.

7. The method of claim 1, wherein the one or more anesthetic agent(s) present in an amount of about 0.01% to about 10.0% w/w.

8. The method of claim 7, wherein the one or more anesthetic agent(s) selected from lidocaine, bupivacaine, prilocaine, procaine, tetracaine, cocaine, ambucaine, amolanone, amylocaine, benoxinate, benzocaine, betoxycaine, biphenamine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dycyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, propanocaine, proparacaine, propipocaine, propoxycaine, pseudococaine, pyrrocaine, ropivacaine, salicyl alcohol, tolycaine, trimecaine, zolamine, and salts thereof.

9. The method of claim 1, further comprising an anti-inflammatory agent(s) such as a steroidal anti-inflammatory agent(s) or non-steroidal anti-inflammatory agent(s), muscle relaxing agent(s), or pharmaceutically acceptable salt thereof.

10. The method of claim 9, wherein the steroidal anti-inflammatory agent(s) is selected from prednisolone, methylprednisolone, triamcinolone acetonide, cortisone, cortisone acetate, dexamethasone, dexamethasone acetate, dexamethasone tertiary butyl acetate, hydrocortisone acetate, prednisolone acetate, betamethasone acetate, betamethasone, fluticasone propionate, budesonide, tipredane, dexamethasone, beclomethasone diproprionate, prednisolone, flucinolone, mometasone furoate, rofleponide palmitate, flumethasone, flunisolide, ciclesonide, deflazacort and non-steroidal anti-inflammatory agent(s) is selected diclofenac, piroxicam, nimesulide, ketoprofen, ibuprofen, and methyl salicylate or a pharmaceutically acceptable salt thereof and the one or more antispasmodic agents is selected from 1,2-butanediol, 1,2-pentanediol, 1,2-hexanediol, 1,2-heptanediol, 1,2-octanediol, 1,2,3-butanetriol, 1,2,3-pentanetriol, 1,2,3-hexanetriol, 1,2,3-heptanetriol, and 1,2,3-octanetriol.

11. The method of claim 1, wherein the pharmaceutical compositions are administered topically in one of a pharmaceutically accepted dosage forms such as gel, cream, lotion, ointment, patch, spray, foam, film forming mixture, emulsion, microemulsion, suspension, poultice, liniment, or tincture.

12. A pharmaceutical composition comprising one or more antispasmodic agent(s) selected from C₄-C₈ aliphatic-1,2-diols and/or C₄-C₈ aliphatic-1, 2, 3-triols and one or more anesthetic agent(s) and one or more pharmaceutically acceptable excipients.

13. The pharmaceutical composition of claim 12, wherein the one or more antispasmodic agent(s) present in an amount of about 0.5% to about 20.0% w/w.

14. The pharmaceutical composition of claim 13, wherein the one or more antispasmodic agent(s) selected from a C₄-C₈ alkane-1,2-diol and/or a C₄-C₈ alkane-1,2,3-triol.

15. The pharmaceutical composition of claim 13, wherein the one or more antispasmodic agent(s) is a C₄-C₈ alkane-1,2-diol.

16. The pharmaceutical composition of claim 13, wherein the one or more antispasmodic agent(s) is a C₄-C₈ alkane-1,2,3-triol.

17. The pharmaceutical composition of claim 15, wherein the one or more antispasmodic agent(s) is a C₄-C₈ alkane-1,2-diol, selected from a 1,2-butanediol, 1,2-pentanediol, 1,2-hexanediol, 1,2-heptanediol, and 1,2-octanediol.

18. The pharmaceutical composition of claim 16, wherein the one or more antispasmodic agent(s) is a C₄-C₈ alkane-1,2,3-triol, selected from 1,2,3-butanetriol, 1,2,3-pentanetriol, 1,2,3-hexanetriol, 1,2,3-heptanetriol, and 1,2,3-octanetriol.

19. The pharmaceutical composition of claim 12, wherein the one or more anesthetic agent(s) present in an amount of about 0.01% to about 10.0% w/w and is selected from lidocaine, bupivacaine, prilocaine, procaine, tetracaine, cocaine, ambucaine, amolanone, amylocaine, benoxinate, benzocaine, betoxycaine, biphenamine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dycyclomine, ecgonidine, ecgonine, ethyl chloride, etidocaine, beta-eucaine, euprocin, fenalcomine, fomocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, propanocaine, proparacaine, propipocaine, propoxycaine, pseudococaine, pyrrocaine, ropivacaine, salicyl alcohol, tolycaine, trimecaine, zolamine, and salts thereof.

20. A method of the treating muscle spasm, wherein the muscle spasm is associated with torticollis or pain after chemotherapy or cervical myofascial pain syndrome, commonly known as tension type headache, the method comprising:
administering, to a patient, a pharmaceutical composition comprising 1,2-Hexanediol present in an amount of about 5% w/w alone or in combination with lidocaine present in an amount of about 4% w/w and one or more pharmaceutically acceptable excipients.
